(19) United States
(12) Patent Application Publication
(10) Pub. No.: US 2014/0222351 A1
(43) Pub. Date: Aug. 7, 2014

(54) DEVICE AND METHOD TO ASSIST TREATMENT OF THE CORNEA

(71) Applicant: Frédéric Hehn, Nancy (FR)

(72) Inventor: Frédéric Hehn, Nancy (FR)

(21) Appl. No.: 14/345,766

(22) PCT Filed: Sep. 18, 2012
(86) PCT No.: PCT/FR2012/000370

§ 371 Date: Mar. 19, 2014

(30) Foreign Application Priority Data
Sep. 19, 2011 (FR) 1102842

(51) Int. Cl. A61B 3/00; G06F 19/00

(52) U.S. Cl. CPC A61B 3/0025; G06F 19/36
USPC 702/19

(57) ABSTRACT

A method and device for assisting treatment of the cornea, the device including a memory (4), an interface (8) capable of receiving and supplying data, a processing unit (6) for calculating and supplying to the interface (8) an ophthalmological value for use in the treatment of the cornea, a scheduler (10) which configured, upon receipt of initial central keratometric index (KC) data and initial asphericity value (QI) data at the interface (8), to store those data in the memory (4), to call the processing unit (6) with the initial central keratometric index (KC) and initial asphericity value (QI) data as well as with target asphericity value (QV) data in order to calculate a peripheral keratometric index (KP), to store the corresponding data in the memory (4) and to supply them to the interface (8).

The invention relates to the field of ophthalmology and more particularly of laser surgery.

The field of the correction of myopia and presbyopia by laser surgery is relatively young and has experienced many findings and advances over the past ten years.

However, this field remains a field which is at the forefront of research and in which the maturity of the methods remains relative. This manifests itself especially in the fact that there is at present no surgery with which both myopia and presbyopia can be corrected satisfactorily.

The invention is going to improve the situation.

To that end, the invention proposes a device to assist treatment of the cornea comprising a memory, an interface capable of receiving and supplying data, as well as a processing unit for calculating and supplying to the interface an ophthalmological value for use in the treatment of the cornea.

The device further comprises a scheduler which configured, upon receipt of initial central keratometric index data and initial asphericity value data at the interface, to store those data in the memory, to call the processing unit with said initial central keratometric index and initial asphericity value data as well as with target asphericity value data in order to calculate a peripheral keratometric index, to store the corresponding data in the memory and to supply them to the interface.

The invention relates also to a method to assist treatment of the cornea, which comprises:
a. receiving initial central keratometric index data and initial asphericity value data,
b. calculating a peripheral keratometric index from the initial central keratometric index and initial asphericity value data as well as from target asphericity value data,
c. storing and supplying corresponding peripheral keratometric index data.

Other features and advantages of the invention will better become apparent upon reading the following description, which refers to examples given by way of illustration and without implying any limitation, and to the drawings, in which:

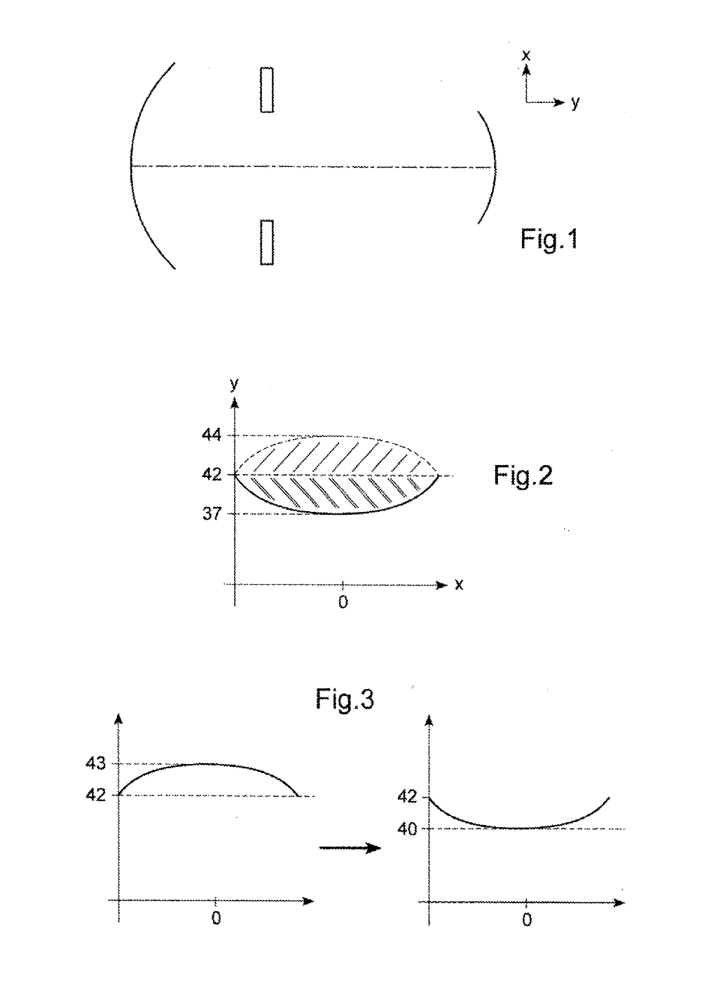

FIG. 1 shows an optical diagram of an eye,

FIG. 2 shows three keratometric profiles of an eye,

FIG. 3 shows a diagram of a method of the prior art,

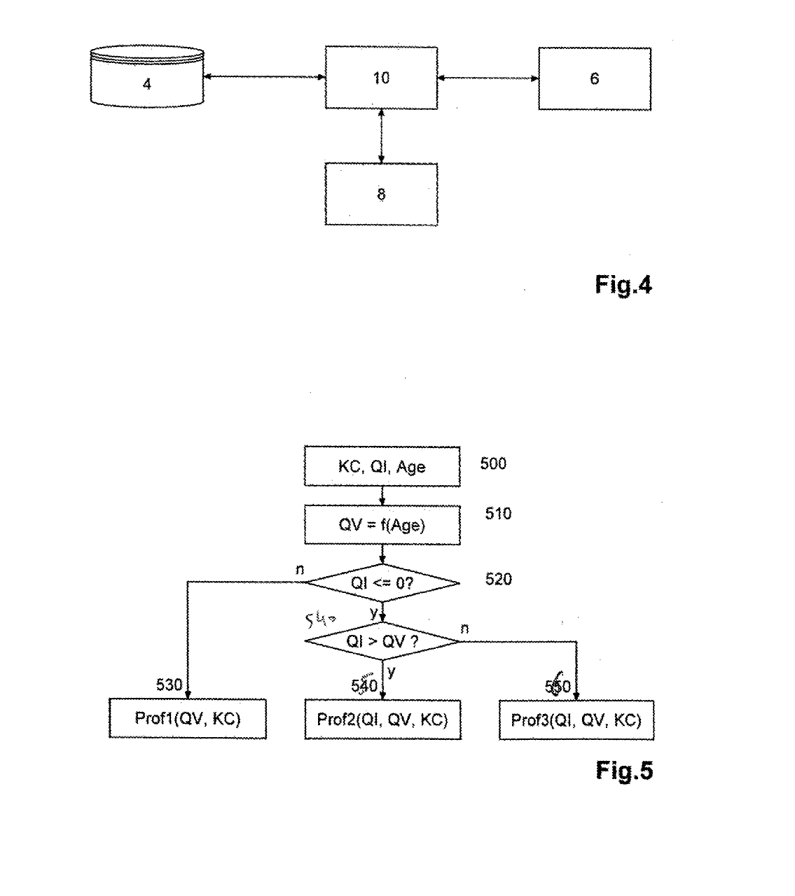

FIG. 4 shows a diagram of a device according to the invention,

FIG. 5 shows a flow diagram of an example of the implementation of a method by the device of FIG. 4,

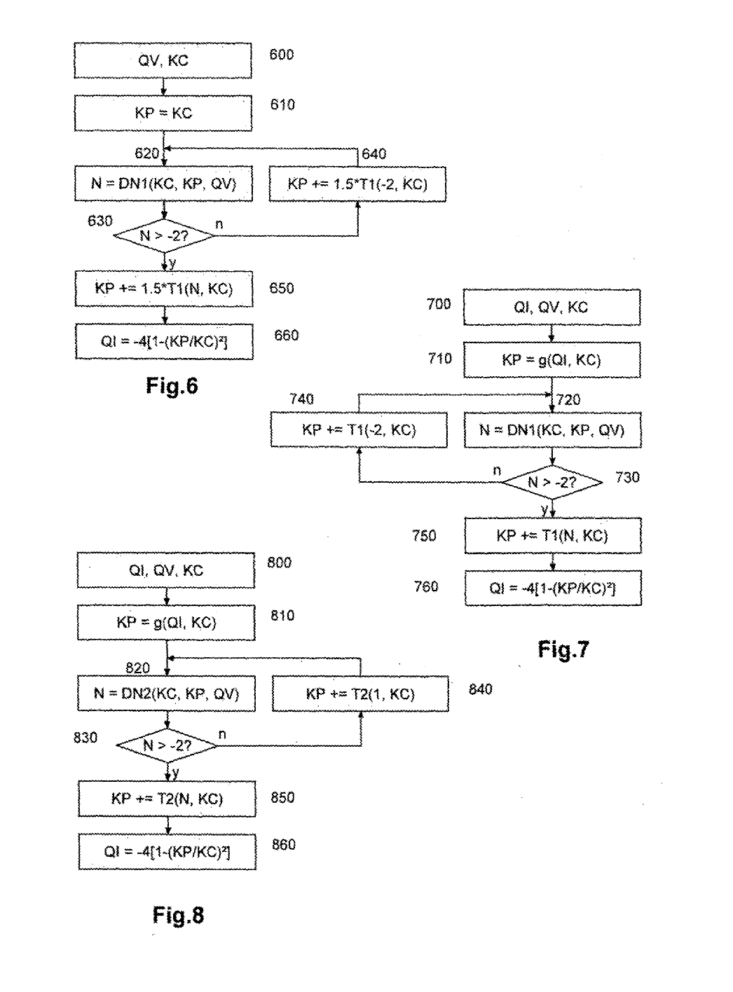

FIGS. 6 to 8 show examples of the implementation of functions of FIG. 5, and

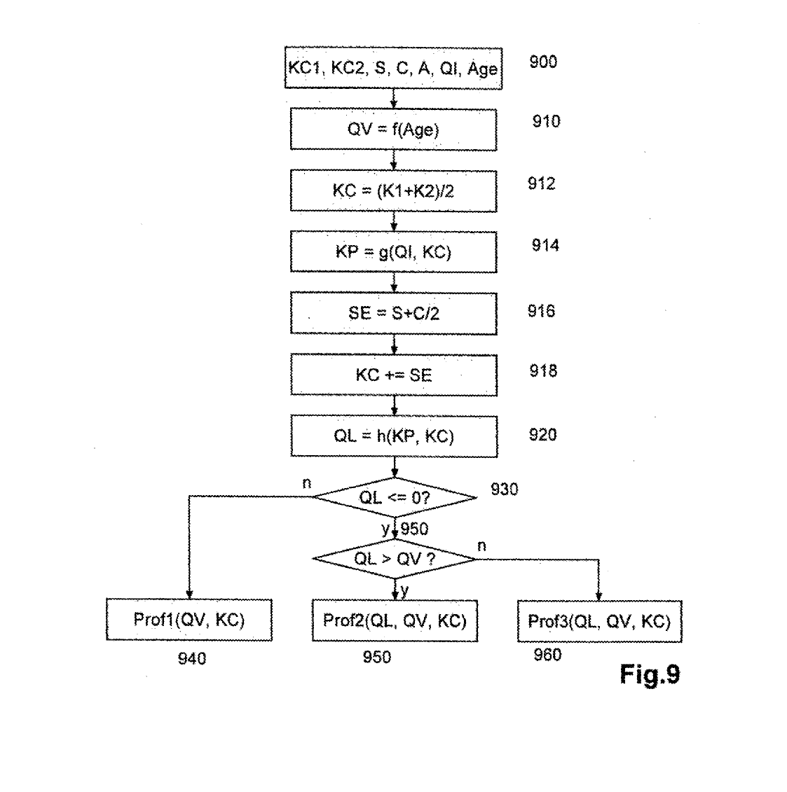

FIG. 9 shows a flow diagram of another example of the implementation of a method by the device of FIG. 4.

The drawings and the description below contain, for the most part, elements of a certain nature. They may therefore not only serve for better understanding of the present invention, but also contribute to the definition thereof, where appropriate.

The nature of the present description is such that it includes elements which are liable to protection by royalties and/or copyright. The holder of the rights has no objection to the identical reproduction by anyone of the present patent document or of its description, as it appears in the official files. For the rest, he reserves his rights wholly.

The detailed description is further supplemented by Annex A, which gives the formulation of some mathematical formulae employed within the context of the invention. This Annex is provided for the purpose of clarification, and to facilitate cross-referencing. It is an integral part of the description and may therefore not only serve for better understanding of the present invention, but also contribute to the definition thereof, where appropriate.

FIG. 1 shows an optical diagram allowing the vision in an eye to be modelled. An eye 12 comprises a cornea 14, an iris 16, a crystalline lens (not shown) and a retina 18.

The cornea 14 acts as a lens which concentrates the light rays, the iris 16 acts as a diaphragm, and the retina 18 acts as a photoreceptor. Ideally, the cornea 14 is prolate and is at a distance from the retina 18 such that all images are formed in a focused manner on the retina 18 (zero spherical aberration).

That is generally not the case. As can be seen in FIG. 2, there are three main types of corneal profile:
the prolate profile, for which the keratometric index is slightly greater at the centre of the eye than at the periphery, which causes asphericity Q<0, with single-line hatching in FIG. 2,
the spherical profile, for which the keratometric index is constant over the eye (Q=0), and
the oblate profile, for which the keratometric index is slightly less at the centre of the eye than at the periphery, which induces asphericity Q>0, with double-line hatching in FIG. 2.

In general, a prolate or slightly hyper-prolate profile is preferred because it allows better near vision. An oblate profile is prejudicial to distance vision, in particular night vision.

Myopia and hypermetropia are two ophthalmological conditions which result in blurred vision. In the case of myopia, the eye is too long and the retina 18 is disposed after the focal plane of the cornea 14. Accordingly, the rays corresponding to distant images are not focused correctly and distance vision is not clear. In the case of hypermetropia, the reverse is true: the eye is too short. In this case, however, the human eye is able to contract to partly compensate for this defect. Another ophthalmological condition is presbyopia.

Myopia can be corrected by laser by altering the keratometric profile of the cornea 14 in order to compensate for the defect of the eye. This is shown, for example, in FIG. 3, in which an eye having a myopia of −6.00 dioptres has been corrected by reducing the keratometric index at the centre of the eye from 43 to 37. The problem of this treatment is that it produces an oblate profile.

This problem is enhanced in older people. From the age of 40, the human eye gradually loses its ability to contract in order to deform the crystalline lens, which is necessary for focusing in near vision.

This is even worse when the eye has an oblate profile. In order to compensate for presbyopia it is possible to add a magnifying lens, but distance vision is then no longer possible. It therefore appears that it is not possible at present to treat both myopia and presbyopia surgically, nor to treat one of the two in isolation without prejudicing either the distance vision or the near vision. At present, the only treatments that exist consist in treating one eye for distance vision and the other eye for near vision. Such treatments produce a “bascule” called monovision. However, this does not give satisfactory results.

The work carried out by the Applicant has led him to study the Zernicke polynomials and their application to the treatment of the conditions explained above. Conventionally, it is believed that only the polynomial C4 is of use, and that all the polynomials of higher order are ineffective.

The Applicant has found that one of these polynomials, more precisely C12, can be combined with the polynomial C4 in order to treat distance vision and near vision simultaneously.

More precisely, the Applicant has found that a corneal profile can be calculated from the polynomial C12, and that this profile allows the problems associated with near vision to be treated without affecting distance vision.

A simplified explanation is that this treatment will produce a corneal profile that is worked mainly at the periphery, with a slightly prolate eye. The asphericity that results is advantageously used to improve the near vision, while the distance vision is not affected since it is mainly at the centre of the eye. This method is called isovision and, unlike monovision, allows each eye to have excellent vision, both distance and near.

This finding goes against all the established prejudices, which state that this treatment is not sufficiently powerful to work, or is at the expense of a loss of distance vision.

FIG. 4 shows a simplified diagram of a calculation device for ophthalmological treatment according to the invention.

The device 2 comprises a memory 4, a processing unit 6, an interface 8 and a scheduler 10.

In the example described here, the memory 4 is a conventional storage medium, which can be a hard disk drive or solid-state drive (SSD), flash or ROM memory, a physical storage medium such as a compact disk (CD), a DVD disk, a Blu-Ray disk, or any other type of physical storage medium. The storage unit 4 can also be remote, on a storage area network (SAN), or on the Internet, or generally in the “cloud”.

In the example described here, the processing unit 6 is a software element executed by a computer containing them. However, it may be executed in a distributed manner on several computers, or it can be in the form of a printed circuit (ASIC, FPGA or the like), or of a dedicated single- or multi-core microprocessor (NoC or SoC).

The interface 8 allows a practitioner to enter the parameters relating to the patient for whom the ophthalmological treatment is desired, and to adjust some of those parameters if necessary. The interface 8 can be electronic, that is to say it can be a link between the device 2 and another apparatus allowing the practitioner to interact with the device 2. The interface 8 can also include such an apparatus and comprise, for example, a display and/or loudspeakers in order to permit communication with the practitioner.

The scheduler 10 selectively controls the processing unit 6 and the interface 8 and accesses the memory 4 in order to carry out the treatments according to the invention.

FIG. 5 shows a flow diagram of a first example of the calculation of a corneal profile carried out by the device of FIG. 3 for treating a defect of near vision.

This treatment begins at an operation 500 by the receiving of parameters KC, QI and Age. These parameters can be pre-stored in the memory 4, for example when they are obtained from previous examinations. They can also be entered manually by means of the interface 8 or received from a measuring apparatus by that same interface or another interface.

The parameter KC represents the average central keratometric index. This index can be obtained, for example, by measuring the average radius of curvature of the cornea along two axes in a central zone having a diameter of 3 mm, and by averaging the values. This type of measurement can give a keratometric profile of the type shown in FIGS. 2 and 3.

The parameter QI represents the initial asphericity of the eye. By producing the keratometric profile it is possible to determine this parameter, which is proportional to the area between the central keratometric index (of value 43, for example) and the measured profile.

Finally, the parameter Age is the age of the person. This parameter is used in an operation 510 to determine the target asphericity QV. To that end, a function f( ) is called with the parameter Age. Determination of the value QV can be taken, for example, from the following table.

| Age | 40-42 | 43-44 | 45-49 | 50-54 | 55-59 | 60-64 | 65-69 | 70+ |
|---|---|---|---|---|---|---|---|---|
| QV | −0.65 | −0.70 | −0.75 | −0.80 | −0.85 | −0.90 | −0.95 | −1 |

Once the parameter QV has been determined, calculation of the corneal profile allowing correction of near vision to be obtained is started.

Accordingly, in an operation 520, a test is carried out on the initial asphericity parameter QI.

When QI is strictly positive, a particular treatment is provided to take account of the fact that the asphericity must be reduced considerably, and a function Prof1( ) is executed in an operation 530. In practice this means that the peripheral corneal index KP is initially greater than the central corneal index KC, and that it will be necessary to “hollow out” the cornea at the periphery and reduce KP in order to obtain a slightly prolate profile.

FIG. 6 shows an example of the implementation of the function Prof1( ). The function Prof1( ) receives as arguments the parameters QI, QV and KC in an initial operation 600.

Then, in an operation 610, the parameter KC is used as the starting value for the value of the peripheral keratometric index of the corneal profile that is to be established. The peripheral keratometric index KP can be measured in a similar manner to the central keratometric index, in a zone having a radius of between 6.5 mm and 8.0 mm of the centre of the eye. It is the specification of this zone which will allow the near vision to be improved.

Then, in an operation 620, a number N is determined from the parameters KC, KP and QV in a function DN1( ) and will be explained below. The resulting number N is comprised between 0 and −2.

A test is then carried out on the number N in an operation 630. In the case where the number N is −2, this means that the calculation of KP will have to be carried out several times. The peripheral index KP is then modified a first time in an operation 640, and then the operation 620 is repeated.

If the number N is greater than −2, that is to say between 0 and −2, then the calculation of KP is carried out by a function T1( ) in an operation 650.

Function T1( ) is based on the use of two parameters N1 and N2, the sum of which is equal to the number N, as is shown in formula [10] in Annex A. Furthermore, function T1( ) also depends on KC. The formula of T1( ) is given with reference [20] in Annex A.

In the example described here, the value of the number N indirectly defines the value of the parameter N1, and the value of the parameter N2 can be derived by subtraction. For example, when N is −1.05, N1 is 0.1 and N2 is −0.95; when N is −0.4, N1 is 0 and N2 is −0.4; N is −1.83, N1 is 0.9 and N2 is −0.93, etc. In general, the relationship between N, N1 and N2 may be defined by a calculation law or by using a lookup table.

Once operation 650 is complete, the final asphericity QF is calculated in an operation 660.

Calculation of the number N is carried out so that the difference between QF and QI is less than 0.01. Accordingly, in order to determine the number N, the range [0; −2] is explored in order to find the suitable number N. The result of this is that operations 640 and 650 as well as 660 can follow from operation 620. In a variant, the calculations described for those operations may likewise be repeated.

In other words, the function DN1( ) determines a number N by scanning the range between 0 and −2, calculating the corresponding peripheral keratometric index KP each time, as well as the corresponding final asphericity, until the target asphericity QV is reached satisfactorily.

If there is no corresponding value in that range, N is fixed equal to −2, KP is modified, and the search is reproduced. This is repeated until an asphericity close to the target asphericity QV is obtained. In practice, two passes are generally sufficient.

Exploration of the range between 0 and −2 can be carried out in many different ways, for example by a linear path, by dichotomy, or by any other algorithm allowing convergence as quickly as possible.

Once operation 530 has been executed, the operator then has both the central corneal index KC and the peripheral corneal index KP and can thus control a laser in order to correct the near vision.

When the initial asphericity is negative, a second test is carried out in an operation 540. In this operation, it is determined whether the initial asphericity QI is greater than or less than the target asphericity QV.

In the first case, slight “hollowing out” at the periphery is again required in order to reduce the asphericity. In the second case, on the other hand, “hollowing out” more at the centre is required, in order to increase it.

In the first case, a function Prof2( ) is executed in an operation 550. An example of the carrying out of the function Prof2( ) is shown in FIG. 7. The function Prof2( ) is very similar to the function Prof1( ) and the operations whose reference numerals bear the same multiple of ten are identical.

Only two elements differ between the function Prof1( ) and the function Prof2( ) Firstly, the index KP is not initialised with the index KC but by means of a function g( ). The function go is defined with formula [50] in Annex A. Calculation of KP then depends on the function T1( ) but with a multiplication factor of 1 for operations 740 and 750 instead of a multiplication factor of 1.5 for operations 640 and 650.

In the second case, a function Prof3( ) is executed in an operation 560. An example of the carrying out of the function Prof3( ) is shown in FIG. 8. The function Prof3( ) is very similar to the function Prof2( ) and the operations whose reference numerals bear the same multiple of ten are identical.

Only two elements differ between the function Prof1( ) and the function Prof2( ) Firstly, the number N is not calculated with the function DN1 but with a function DN2 in operation 820. This is due to the fact that the calculation of KP in operations 840 and 850 is carried out with a function T2( ). The formula of the function T2( ) is given in formula [60] in Annex A. Function Prof3( ) then differs in that the number N is in a range between 0 and 1 and not between 0 and −2.

FIG. 9 shows another example of the carrying out of FIG. 5. In this embodiment, the corneal profile is calculated in order to correct both near vision and distance vision.

To that end, in a first operation 900 parameters KC1, KC2, S, C, A, QI and Age are received.

The parameters KC1 and KC2 are central keratometric indices according to two orthogonal axes, the parameter S is the sphere, the parameter C is the cylinder, and the parameter A is the axis.

These parameters can be pre-stored in the memory 4, for example when they are obtained from previous examinations. They can also be entered manually by means of the interface 8 or received from a measuring apparatus by that same interface or another interface.

The target asphericity QV is then calculated in an operation 910 which is identical to operation 510.

The central keratometric index KC is then calculated in an operation 912 by averaging the keratometric indices KC1 and KC2, and then the keratometric index KP is calculated in an operation 914 which is identical to operations 720 and 820.

The spherical equivalent SE is then determined in an operation 916, and then the central keratometric index KC permitting correction of the distance vision is calculated in an operation 918 by adding the spherical equivalent of operation 916.

Once those operations have been carried out, calculation of the peripheral keratometric index KP for the near vision can be calculated in the same manner as for FIG. 5, and operations 920 to 960 are identical to operations 520 to 560, except that it is no longer necessary to calculate KP in the functions Prof2( ) and Prof(3).

Once all those operations have been carried out, a laser treatment can be carried out with KC, and the axis A for the distance vision, and with KP for the near vision.

It is clear from the above that the Applicant has found a treatment which is based on the definition of a corneal profile which concerns both the centre and the periphery of the cornea.

The aim is to obtain a slightly prolate corneal profile, the asphericity of which allows near vision to be improved. Since this profile is produced on distinct parts of the cornea, correction of near vision and of distance vision can be obtained on the same eye.

Accordingly, a peripheral keratometric index is calculated, and optionally a central keratometric index, and those indices can be used to carry out a surgical treatment, for example laser treatment, in order to correct the near vision and optionally the distance vision.

Moreover, it appears that these calculations may also be suitable for the calculation of a lens profile. Since the lens comeson the cornea, the invention described here may be used to define the shape thereof, with the same results.

The device described here can be integrated into a laser apparatus for ophthalmic surgery or into an apparatus for the manufacture of ophthalmological lenses, or it can be separate from such apparatuses.

In different variants, the device may have the following features:
the peripheral keratometric index (KP) is calculated from a polynomial of order 2 in which the initial central keratometric index (KC) is the variable,
the polynomial of order 2 depends on the sign of the initial asphericity value (QI) and/or on the sign of the difference between the initial asphericity value (QI) and the target asphericity value (QV),
the target asphericity value (QV) is determined by the processing unit (6) from age (Age) data received at the interface and/or stored in the memory (4),
the scheduler (10) is further configured to receive primary and secondary central keratometric index (K1, K2) data, sphere and cylinder (S, C) data, to call the processing unit (6) with those data in order to calculate a modified central keratometric index (KC) and an intermediate asphericity value (QL), and to calculate the peripheral keratometric index (KP) by calling the processing unit (6) with the modified central keratometric index (KC) and intermediate asphericity value (QL) data as well as with target asphericity value (QV) data.

In different variants, the method may have the following features:
step b. comprises the use of a polynomial of order 2 in which the initial central keratometric index (KC) is the variable,
the polynomial of order 2 depends on the sign of the initial asphericity value (QI) and/or on the sign of the difference between the initial asphericity value (QI) and the target asphericity value (QV),
the target asphericity value (QV) is determined by the processing unit (6) from age (Age) data received in step a.,
step a. comprises the receiving of primary and secondary central keratometric index (K1, K2) data and of sphere and cylinder (S, C) data, wherein those data (K1, K2, S, C) are used to calculate a modified central keratometric index (KC) and an intermediate asphericity value (QL), and wherein step b. uses the modified central keratometric index (KC) and intermediate asphericity value (QL) data as well as target asphericity value (QV) data to calculate the peripheral keratometric index (KP).

### ANNEX A

N=N1+N2 [10]

T1(KC,N)=N1*TS2(KC)−N2*TS1)(KC) [20]

TS1(KC)=−0.003673*KC²+0.160196*KC−2.2675 [30]

TS2(KC)=−0.004174*KC²+0.192788*KC−2.8146 [40]

KP=(\sqrt{(Q1+4)})*KC/2 [50]

T2(KC,N)=−N1*TS3(KC)−N2*TS1(KC) [60]

TS3(KC)=0.00317*KC²−0.1345*KC+1.909 [70]

1. Device for assisting treatment of the cornea, comprising a memory (4), an interface (8) capable of receiving and supplying data, as well as a processing unit (6) for calculating and supplying to the interface (8) an ophthalmological value for use in the treatment of the cornea, characterised in that it further comprises a scheduler (10) which configured, upon receipt of initial central keratometric index (KC) data and initial asphericity value (QI) data at the interface (8), to store those data in the memory (4), to call the processing unit (6) with said initial central keratometric index (KC) and initial asphericity value (QI) data as well as with target asphericity value (QV) data in order to calculate a peripheral keratometric index (KP), to store the corresponding data in the memory (4) and to supply them to the interface (8).

2. Device according to claim 1, wherein the processing unit (6) is configured to calculate the peripheral keratometric index (KP) from a polynomial of order 2 in which the initial central keratometric index (KC) is the variable.

3. Device according to claim 2, wherein the processing unit (6) is configured to calculate the peripheral keratometric index (KP) from a polynomial of order 2 which depends on the sign of the initial asphericity value (QI) and/or on the sign of the difference between the initial asphericity value (QI) and the target asphericity value (QV).

4. Device according to claim 1, wherein the processing unit (6) is configured to calculate the target asphericity value (QV) from age (Age) data received at the interface and/or stored in the memory (4).

5. Device according to claim 1, wherein the scheduler (10) is further configured to receive primary and secondary central keratometric index (KC1, KC2) data, sphere and cylinder (S, C) data, to call the processing unit (6) with those data in order to calculate a modified central keratometric index (KC) and an intermediate asphericity value (QL), and to calculate the peripheral keratometric index (KP) by calling the processing unit (6) with the modified central keratometric index (KC) and intermediate asphericity value (QL) data as well as with target asphericity value (QV) data.

6. Calculation method to assist treatment of the cornea, characterised in that it comprises:
a. receiving initial central keratometric index (KC) data and initial asphericity value (QI) data,
b. calculating a peripheral keratometric index (KP) from the initial central keratometric index (KC) and initial asphericity value (QI) data as well as from target asphericity value (QV) data,
c. storing and supplying corresponding peripheral keratometric index (KP) data.

7. Method according to claim 6, wherein step b. comprises the use of a polynomial of order 2 in which the initial central keratometric index (KC) is the variable.

8. Method according to claim 7, wherein the polynomial of order 2 depends on the sign of the initial asphericity value (QI) and/or on the sign of the difference between the initial asphericity value (QI) and the target asphericity value (QV).

9. Method according to claim 6, wherein the target asphericity value (QV) is determined by the processing unit (6) from age (Age) data received in step a.

10. Method according to claim 6, wherein step a. comprises the receiving of primary and secondary central keratometric index (KC1, KC2) data and of sphere and cylinder (S, C) data, wherein those data (KC1, KC2, S, C) are used to calculate a modified central keratometric index (KC) and an intermediate asphericity value (QL), and wherein step b. uses the modified central keratometric index (KC) and intermediate asphericity value (QL) data as well as target asphericity value (QV) data to calculate the peripheral keratometric index (KP).

11. Method according to claim 7 wherein the target asphericity value (QV) is determined by the processing unit (6) from age (Age) data received in step a.

12. Method according to claim 8, wherein the target asphericity value (QV) is determined by the processing unit (6) from age (Age) data received in step a.

13. Method according to claim 7, wherein step a. comprises the receiving of primary and secondary central keratometric index (KC1, KC2) data and of sphere and cylinder (S, C) data, wherein those data (KC1, KC2, S, C) are used to calculate a modified central keratometric index (KC) and an intermediate asphericity value (QL), and wherein step b. uses the modified central keratometric index (KC) and intermediate asphericity value (QL) data as well as target asphericity value (QV) data to calculate the peripheral keratometric index (KP).

14. Method according to claim 8, wherein step a. comprises the receiving of primary and secondary central keratometric index (KC1, KC2) data and of sphere and cylinder (S, C) data, wherein those data (KC1, KC2, S, C) are used to calculate a modified central keratometric index (KC) and an intermediate asphericity value (QL), and wherein step b. uses the modified central keratometric index (KC) and intermediate asphericity value (QL) data as well as target asphericity value (QV) data to calculate the peripheral keratometric index (KP).

15. Device according to claim 2, wherein the scheduler (10) is further configured to receive primary and secondary central keratometric index (KC1, KC2) data, sphere and cylinder (S, C) data, to call the processing unit (6) with those data in order to calculate a modified central keratometric index (KC) and an intermediate asphericity value (QL), and to calculate the peripheral keratometric index (KP) by calling the processing unit (6) with the modified central keratometric index (KC) and intermediate asphericity value (QL) data as well as with target asphericity value (QV) data.

16. Device according to claim 3, wherein the scheduler (10) is further configured to receive primary and secondary central keratometric index (KC1, KC2) data, sphere and cylinder (S, C) data, to call the processing unit (6) with those data in order to calculate a modified central keratometric index (KC) and an intermediate asphericity value (QL), and to calculate the peripheral keratometric index (KP) by calling the processing unit (6) with the modified central keratometric index (KC) and intermediate asphericity value (QL) data as well as with target asphericity value (QV) data.

17. Device according to claim 4, wherein the scheduler (10) is further configured to receive primary and secondary central keratometric index (KC1, KC2) data, sphere and cylinder (S, C) data, to call the processing unit (6) with those data in order to calculate a modified central keratometric index (KC) and an intermediate asphericity value (QL), and to calculate the peripheral keratometric index (KP) by calling the processing unit (6) with the modified central keratometric index (KC) and intermediate asphericity value (QL) data as well as with target asphericity value (QV) data.
